(19) United States
(12) Patent Application Publication
(10) Pub. No.: US 2020/0405930 A1
(43) Pub. Date: Dec. 31, 2020

(54) OPTICAL SENSOR ASSEMBLY IN CATHETER-BASED MEDICAL DEVICES

(71) Applicant: ABIOMED, Inc., Danvers, MA (US)

(72) Inventors: Tao Zhang, Danvers, MA (US); Zenghong Tao, Danvers, MA (US); Soumen Das, Danvers, MA (US)

(21) Appl. No.: 16/913,040

(22) Filed: Jun. 26, 2020

Related U.S. Application Data
(60) Provisional application No. 62/868,527, filed on Jun. 28, 2019.
(60) Provisional application No. 62/868,517, filed on Jun. 28, 2019.

(51) Int. Cl. A61M 1/10; A61M 1/36; A61M 1/12

(52) U.S. Cl. CPC A61M 1/1086; A61M 1/1031; A61M 1/3659; A61M 2205/0244; A61M 2207/10; A61M 2205/3306; A61M 1/125

(57) ABSTRACT

An optical sensor assembly for use in a blood pump assembly comprising a visor affixed to a pump housing of the blood pump assembly. A support jacket is in contact with an inner surface of the visor, and defines a cavity in which an optical sensor is disposed. A silicone composition is introduced into the cavity, where it cures. The silicone composition within the cavity protects the optical sensor, and the support jacket prevents the overflowing of the silicone composition and contamination of the visor. The silicone composition comprises a silicone component and a plasticizer with a silicone to plasticizer ratio selected to provide one or more of the desired rigidity, tackiness, adhesion strength, viscosity, shelf life, pot life, and curing properties. The silicone composition may comprise more than one silicone component. A method of manufacturing the optical sensor assembly and the silicone composition.

### CROSS-REFERENCE TO RELATED APPLICATIONS

This application claims the benefit of U.S. Provisional Application No. 62/868,517, filed Jun. 28, 2019, and U.S. Provisional Application No. 62/868,527, filed Jun. 28, 2019, the disclosure of both of which are incorporated by reference herein in their entirety.

### BACKGROUND

An intravascular blood pump assembly, such as an assembly with an intracardiac blood pump, may be introduced into the heart to deliver blood from the heart into an artery. Intravascular blood pumps can be introduced percutaneously during a cardiac procedure through the vascular system, such as by a catheterization procedure. Some blood pumps are designed to support the left side of the heart, where they pull blood from the left ventricle of the heart and expel the blood through a cannula into the aorta. Some blood pumps that support the left side of the heart are introduced by a catheterization procedure through the femoral artery, into the ascending aorta, across the aortic valve, and into the left ventricle. Some systems are designed to support the right side of the heart, where the blood pump is introduced through a vein and into the right side of the heart through the venous system (i.e., the vena cava). Blood pump systems may also be surgically implanted or inserted through the subclavian and/or carotid arteries. During the insertion of a blood pump assembly into a patient through a blood vessel, it may be difficult to advance the blood pump through the tortuous paths or calcified anatomy of the patient.

Complications involving the introduction of the pump due to these tortuous paths may, in some cases, cause damage to the blood pump assembly, or to the patient. For example, the blood pump or its components may be damaged or may damage the vasculature of a patient during insertion or operation. Components of the blood pump may detach from the pump during the introduction and operation, for example due to the shear forces exerted on the blood pump components by the vasculature, or the blood. A damaged blood pump may need to be removed or replaced, or it may no longer be accurate, or operational. For example, damage to pump sensors may prevent accurate pump introduction or operation.

The blood pump's sensors (e.g. an optical sensor) can be particularly vulnerable to damage during insertion or operation of the pump. For example, the shear forces exerted on such an optical sensor deployed with a blood pump within a patient can cause the sensor to crack. Additionally, these shear forces can at least partially dissolve, erode or scratch the sensor membrane. The incorporation of dissolved silicone particles may be detrimental to the health of the patient. In other situations, the optical sensor or its components may become separated from the rest of the system, such as from the blood pump's housing. Damage to the optical sensor can prevent the sensor from conveying to the practitioner the important signals picked up by the sensor. Similarly, the separation of components of the blood pump within the vasculature of the patient can negatively impact patient health.

One approach protects the optical sensor by a single layer of cured silicone gel applied to the surface of the sensor. Additional layers of silicone gel provide increased protection. However, silicone gel is hydrophobic and therefore can become unstable and overflow because of the capillary pressure exerted on the sensor during operation. The overflowing of the silicone gel impairs the adhesion of the optical sensor to the pump housing. Insufficient adhesion between the optical sensor and the pump housing may cause the device to break within the patient due to the exertion of shear forces on the assembly by blood. Additionally, the overflowing of silicone gel can cause contamination of various components in the area of the pump housing.

In order to protect the measuring surface of the optical sensor (e.g. a diaphragm of the optical sensor) from the forces exerted by blood on the measuring surface during insertion and operation of the blood pump assembly, the measuring surface of the optical sensor is coated with a layer of cured silicone. Due to the unique conditions in which a blood pump assembly comprising an optical sensor is deployed, there are several mechanical properties that must be considered in the determination of an appropriate silicone composition for application to the measuring surface of the optical sensor. The silicone should have the appropriate viscosity, adhesion strength, hardness and tackiness, while being biocompatible to ensure the safety of the patient. The desired mechanical properties are unique to silicone compositions for use in blood pump assemblies, as the conditions in which the pump assemblies operate are unique themselves. Specifically, these mechanical properties allow the composition to be easily handled in manufacturing. Additionally, such mechanical properties allow the composition to flow within the confines of a support jacket of the optical sensor assembly without contaminating the visor of said assembly. Further, a composition having these properties provides the measuring surface of the optical sensor with sufficient protection upon insertion of the blood pump assembly into the patient. Additionally, these unique silicone mechanical properties are preferably attained without disruption to the manufacturing process. Specifically, the mechanical properties advantageously provide protection to the measuring surface of the optical sensor without lengthening the curing process. Additionally, the silicone should have a desired shelf life and pot life.

Thus, it would be desirable to have an improved optical sensor assembly that provides one or more of the advantages of protecting the sensor during pump insertion, facilitating adhesion of the sensor to the pump housing, preventing contamination of the optical sensor assembly components and preventing silicone gel (or other bonding components) from undesirably overflowing when the blood pump assembly is deployed within the patient. Further, it would be desirable to provide ease of manufacturing of such an improved optical sensor assembly when incorporating it into existing blood pump assemblies without impeding or delaying the manufacturing process. Additionally, it is desirable to create a composition that has the desired mechanical properties for the protection of an optical sensor for use in a blood pump assembly and that also can be incorporated into existing methods of manufacturing.

### BRIEF SUMMARY

The systems, methods, and devices described herein provide an optical sensor assembly with an improved sensor protection system for use in an intravascular blood pump system (or other blood pump systems). The blood pump systems have a blood pump (e.g. an Impella® pump) with a sensor system including a sensor (such as an optical sensor) that sits within a support jacket attached or positioned relative to the blood pump. When in use, the sensor aids in positioning the pump and monitoring pump performance and impact. The blood pump may have a rotor inside a pump housing, a cannula that receives blood being pumped through the system, a delivery mechanism (such as a catheter or surgical delivery set) for inserting the pump in the patient, and a drive unit for powering the pump. The drive unit may be an external motor and an electrical connection. Alternatively, the drive unit may be a mechanical cable connecting the rotor to an external motor. The cannula extends distal of the pump, and may include a flexible, atraumatic projection extending distal of the cannula.

The optical sensor assembly is selected to work with the pump and may include an optical sensor seated within a support jacket that is, in turn, affixed or otherwise secured relative to the pump. The support jacket may be configured with a crevice or other cavity that receives the sensor. The support jacket may comprise a polymer or a metal. For example, the support jacket may comprise polyimide or stainless steel. The support jacket may be formed of a polymer or other material that can adhere or be positioned with respect to the pump (or one of its components) so as to support the sensor. For example, the sensor is positioned close enough to the pump to be useful in monitoring pump performance. The support jacket may be configured in the shape of a tube, or any suitable elongate body having an inner surface, an outer surface, and defining a cavity. The support jacket may have a circular, rectangular, ovate, or elliptical cross-section. In some adaptations, the support jacket's cavity is configured to contain an amount of silicone composition or other material sufficient for securing the sensor within the cavity. For example, the amount of silicone composition or other material fills the cavity in which the sensor is located, and surrounds the sensor. A visor may be included over the outer surface of the support jacket to further shield the optical sensor. In some implementations, the visor is in contact with the outer surface of the support jacket. In other implementations, there is a space between the visor and the outer surface of the support jacket. In certain implementations, portions of the visor are in direct contact with the outer surface of the support jacket while other portions of the visor are separated by a space from the outer surface of the support jacket.

According to a first implementation, an optical sensor assembly comprises a visor, a support jacket (for example, a polymer tube or another elongate body having an inner and outer surface and defining a cavity), an optical sensor, and silicone composition. The inner surface of the visor is placed around the support jacket (e.g., the polymer tube) so as to be in contact with the outer surface of the jacket (e.g., the surface of the polymer tube) and shield the sensor positioned within the cavity. In certain adaptations, the support jacket (e.g. polymer tube or metal tube) defines a cavity within its frame. The optical sensor of the assembly is disposed within the cavity. The inner surface of the optical sensor is in contact with the inner surface of the jacket. For example, the inner surface of the optical sensor may be glued to the inner surface of the jacket. In other examples, the inner surface of the optical sensor may be fused to the inner surface of the jacket.

In some implementations, the inner surface of the visor covers at least partly the outer surface of the jacket (e.g., the polymer tube). In further implementations, the inner surface of the visor surrounds a portion of the jacket such that the portion that remains uncovered by the visor is not in contact with the pump housing. In other implementations, the inner surface of the visor surrounds a portion of the jacket such that the portion that remains uncovered by the visor is in contact with the pump housing. In some implementations, the jacket is glued to the visor. In other implementations, the jacket is fused to the visor. In certain implementations, the jacket is fused to the pump housing. In some implementations, the jacket is glued to the pump housing. At least one layer of silicone composition or another similar material (e.g. a platinum-based silicone) coats the outer surface of the optical sensor and fills the cavity defined by the jacket. The silicone composition serves to protect the optical sensor of the blood pump assembly from the shear forces exerted by blood on the optical sensor during the percutaneous insertion and operation of the pump within the patient. Additional layers of silicone composition or another similar material offer additional protection to the sensor. In some embodiments, the silicone composition is a silicone gel.

The jacket (e.g., the polymer tube) may be any one of a variety of polymers or other similar materials. In some implementations, the polymer comprises polyimide. The polymer jacket may be constructed from a blend of polymers. In some implementations, the polymer blend includes polyimide and one or more other polymers. The particular polymer which the polymer jacket comprises is selected to allow for ease of handling. Further, the support jacket may comprise a metal that similarly provides ease of handling. For example, the metal may be stainless steel. In other implementations, the metal is Nitinol. The visor may comprise a metal, a plastic, or a composite material. In certain implementations, the visor may be stainless steel. In other implementations, the metal comprises an alloy. In some examples the alloy is Nitinol. In other implementations, the alloy is a ferrous alloy. In certain implementations, the plastic is polyurethane. The polyurethane may comprise polyether or polyester.

Further, the inner surface of the visor may be configured to bond to the pump housing. In some implementations, the inner surface of the visor is bound to the pump housing by a glue. The glue forms a bond between the visor and the pump housing that can withstand the shear forces exerted by the blood. In certain implementations, the glue is an epoxy. For example, the glue may be a two-part epoxy, or a UV light-bonded epoxy. In other implementations, the inner surface of the visor is fused directly to the pump housing.

The silicone composition may be applied to the optical sensor within the cavity of the jacket. In some embodiments, the silicone composition is a silicone gel. The inner surface of the jacket (e.g., the polymer tube) constrains the flow of the silicone composition such that the silicone composition remains within the cavity and a volume of silicone composition can surround the sensor without overflowing or having to be added and cured one layer at a time. That ability to add silicone composition volumetrically provides the sensor with a thicker layer of protection and permits the silicone composition (or other binding material) to be cured within the cavity in a single step, rather than using a layer-by-layer approach. Thus, the polymer tube allows additional silicone composition to protect the optical sensor without slowing down the manufacturing process.

The cavity within the jacket (e.g., the polymer tube) may be structured to have a range of lengths or radii. In some implementations, the cavity has a length between about 1 centimeter and about 5 centimeters. In other implementations, the cavity has a length between about 2 centimeters and about 4 centimeters. In certain implementations, the cavity has a length of about 3 centimeters. In certain implementations, the cavity has a radius between about 0.1 millimeter and about 0.25 millimeters. In some implementations, the cavity has a radius between about 0.15 millimeters and about 0.20 millimeters. In further implementations, the cavity has a radius of about 0.175 millimeters. At least one advantage of having a polymer tube or other cavity with different acceptable lengths is that tubes of different length can accommodate different amounts of silicone composition, all of which offer additional protection to the optical sensor.

In general, applying the silicone composition or other binding material in the cavity, with the thicker layer of silicone composition, can provide stronger protection for the sensor system. That feature can be helpful in some procedures where the system experiences high shear forces. Such procedures can be completed with optical sensor assemblies having larger polymer tubes, which accommodate larger amounts of silicone composition to provide the sensor with additional protection. The size cavity and volume of silicone composition can be adjusted as needed, for a given patient anatomy. For example, some femoral insertions of blood pumps into obese patients exert greater forces on the blood pump because the blood vessel is deeper with respect to the insertion point than in a patient of a healthy bodyweight. In that case, the cavity size and silicone composition filling level can be set so as to increase the strength of the bond and protection of the optical sensor components of the blood pump, by, for example, increasing the length of the support jacket to create a larger cavity volume. The larger cavity volume can then accommodate a larger volume of silicone composition, which protects the optical sensor from the large shear forces. Cardiac procedures in pediatric patients having smaller anatomies can be completed with optical sensor assemblies having smaller polymer tubes, which allow for the application of additional layers of silicone to the surface of the optical sensor while simultaneously minimizing damage done unto the smaller vasculatures of pediatric patients by larger pumps.

In some implementations, the optical sensor assembly includes a visor configured to interface with the polymer tube or other jacket. The visor is configured to surround the support jacket in order to protect the sensor within the cavity defined by the jacket. The visor provides the jacket with protection from the shear forces exerted on the optical sensor by the blood of a patient during the insertion and operation of the pump into the patient. The visor is attached to the housing with a bond of sufficient strength to withstand the shear forces exerted on the optical sensor assembly during insertion.

The visor and jacket each have inner and outer surfaces; in various adaptations the inner surface of the visor is configured to be in contact with the outer surface of the jacket (e.g., the outer surface of the polymer tube). The cavity may be defined within the perimeter of the jacket (e.g. within the perimeter of a polymer tube, or within the inner surface of an elongate body defining a cavity) and sized to receive the optical sensor. The cavity is configured to be filled with a silicone composition to protect the optical sensor disposed within the cavity. In some implementations, the optical sensor is a silicone optical sensor. The polymer tube further shields the silicone composition from the visor to reduce contamination of the outer surface of the visor. The size of the cavity and the amount of the silicone composition are configured to protect the optical sensor from damage due to forces exerted on the optical sensor during the percutaneous insertion of the blood pump assembly into a patient.

In some implementations, the visor surrounds the support jacket and is anchored to a component of the pump assembly. In certain implementations, the visor is anchored to the pump housing. The visor may be glued to the pump housing, or, in some implementations, fused to the pump housing. The visor comprises a material that provides a sufficiently strong bond between the visor and the pump housing. In some implementations, the visor comprises a metal. The metal may comprise stainless steel, or another similar material. The visor must further comprise a material that provides sufficient protection to the support jacket.

In another implementation, a method of manufacturing a packaging for an optical sensor for use in a blood pump assembly comprises placing an optical sensor within a cavity of a support jacket, such as within a polymer tube that defines a cavity. The method further comprises filling a portion of the cavity with a material, (e.g., silicone composition) and curing the material. The method then comprises surrounding a portion of the support jacket (e.g., a portion of the polymer tube) with a visor, and binding an inner surface of the visor to a blood pump, e.g., to the pump housing of a blood pump. In certain implementations, between about 30 percent and about 90 percent of the cavity is filled with silicone composition. In other implementations, between about 50 percent and about 70 percent of the cavity is filled with silicone composition. In further implementations, about 60 percent of the cavity is filled with silicone composition. The portion of the volume of the cavity that is filled with silicone composition can be selected in order to yield the desire protection to the optical sensor and also to yield a desired manufacturing time, as larger volumes of silicone composition require longer curing times. In some embodiments, the silicone composition is a silicone gel. In some implementations, the optical sensor is a silicone optical sensor. In other implementations, the support jacket (e.g., the polymer tube) comprises polyimide. In certain implementations, the visor comprises a metal. The metal may comprise stainless steel. The material from which the visor is formed is selected in order to yield specific mechanical properties of the visor. In some implementations, the visor is bound to the pump housing by a glue. In certain implementations, glue may be a two-part epoxy. In other implementations, the glue may be a UV light-bonded epoxy. In further implementations, the visor is configured to be fused to the pump housing. The means by which the visor is attached to the pump housing are selected in order to ensure sufficient adhesion strength of the bond between the visor and the pump housing, and such that the adhesion strength allows the bond between the visor and the pump housing to withstand the shear forces exerted by blood on the pump assembly during the insertion and operation of the pump assembly.

The systems, methods, and devices described herein also provide a silicone composition for use in a sensor assembly, such as an optical sensor assembly. An example optical sensor assembly may be configured for use in a blood pump assembly. In general, a blood pump assembly comprises a blood pump having a rotor with a pump housing surrounding one or more blades, and a drive unit. A cannula extends distal of the pump housing, and a flexible, atraumatic projection extends distal of the cannula. The blood pump assembly further comprises an optical sensor assembly. In order to accommodate the environment in which the blood pump assembly operates, a silicone composition is placed over the optical sensor. The composition comprises a silicone component and a plasticizer having a ratio selected to provide one or more of the desired rigidity, tackiness, adhesion strength, viscosity, shelf life, pot life, and curing properties. The composition may include the silicone and plasticizer at a mass or mole ratio. The values of the properties corresponding to this composition are configured to protect the optical sensor without compromising the ability of the sensor to take accurate measurements from within the patient. In some implementations, the composition comprises more than one silicone component. In such implementations, the components may be mixed in a sequence to prevent undesirable residual reactions from occurring. In a first implementation, a method of manufacturing a silicone composition for use in an optical sensor assembly comprises first mixing a first silicone component and a plasticizer to form a first silicone mixture. Subsequently, a second silicone component is mixed with the plasticizer to form a second silicone mixture. The first silicone mixture is then combined with the second silicone mixture to yield the silicone composition, which is configured to protect a measuring surface of the optical sensor. The optical sensor assembly may be suitable for use in a blood pump assembly, and the silicone composition suitable to protect the sensor from shear forces exerted during the insertion and operation of the blood pump assembly within the patient. The composition may be vacuum degassed. Generally, each of the silicone components may be biocompatible.

In some embodiments, the first silicone component comprises an activator. In certain implementations, the activator comprises fumed silica. In certain embodiments, the second silicone component has a catalyst, such as a metallic (e.g., platinum-based) catalyst. In other embodiments, the catalyst is rhenium-based. In some embodiments, the catalyst has an organometallic compound configured to increase the compatibility of the catalyst and the silicone components. In some embodiments, the first silicone component and the plasticizer are different materials. In some embodiments, the first silicone component, the second silicone component, and the plasticizer are different from each other. In some embodiments, at least one of the first silicone component and the plasticizer is NuSil MED4088. In further embodiments, both the first silicone component and the plasticizer are NuSil MED4088. In some embodiments, the plasticizer is a silicone oil plasticizer. In certain embodiments, the plasticizer is NuSil MED360.

The concentrations of the first and second silicone components and the plasticizer may be selected so that the ratios of those components within the composition are at a desired level. For example, the ratio of the first silicone component to the plasticizer and the ratio of the second silicone component to the plasticizer may be selected such that the silicone composition has desired mechanical properties and a desired final ratio within the composition. Specifically, the composition would have at least the desired viscosity, sufficient adhesion strength and tackiness to adhere to the optical sensor, and the composition may advantageously have a sufficient rigidity to allow for ease of handling. An example plasticizer is silicone oil, which decreases the viscosity of the first silicone component and the second silicone component, such that the composition has one or more of the desired mechanical properties identified above, and such that it is easy to handle during manufacturing. The application of excess silicone oil, however, undesirably increases the length of time over which the composition must be cured.

As such, the specific ratios between the components, as well as the mechanical properties of the final composition, may be advantageously selected in order to both protect the optical sensor from the shear forces exerted on the sensor during insertion and use, and allow for efficient handling and manufacturing times.

In some implementations, the ratio of the first silicone component to the plasticizer is between about 1:4 and about 4:1. In certain implementations the ratio of the second silicone component to the plasticizer is between about 1:4 and about 4:1. In further implementations, the ratio of the first silicone component to the plasticizer is between about 1:3 and about 3:1. In certain implementations, the ratio of the second silicone component to the plasticizer is between about 1:3 and about 3:1. In some implementations, the ratio of the first silicone component to the plasticizer is between about 1:2 and about 2:1. In certain implementations, the ratio of the second silicone component to the plasticizer is between about 1:2 and about 2:1. In some implementations, the ratio of the first silicone component to the plasticizer is about 1:1. In further implementations, the ratio of the second silicone component to the plasticizer is about 1:1.

In certain implementations, the ratio of the first silicone component to the second silicone component to the plasticizer of the final composition is between about 1:1:8 and about 2:2:1. In further implementations, the ratio of the first silicone component to the second silicone component to the plasticizer of the final composition is between about 1:1:6 and about 3:3:2. In certain implementations, the ratio of the first silicone component to the second silicone component to the plasticizer of the final composition is between about 1:1:4 and about 1:1:1. In further implementations, the ratio of the first silicone component to the second silicone component to the plasticizer of the final composition is about 1:1:2.

The plasticizer may be added separately to each of the first silicone component and the second silicone component, so as to avoid undesired reaction between the plasticizer and the first and second silicone components. For example, the separate addition of the plasticizer to the first and second silicone components avoids the undesirable interaction between two or more of the components, which could result in an inhomogeneous mixture. As discussed below, this separate addition of the plasticizer to the first and second silicone components may further help attain a desired viscosity of the silicone composition. A composition configured with the desired viscosity may optimally protect the optical sensor during the insertion and operation of the blood pump assembly, and also may allow for ease of handling of the composition in manufacturing.

The mechanical properties of the silicone composition may be selected such that the composition is suitable for use in an optical sensor assembly for a blood pump. For example, the adhesion strength of the silicone composition may be selected so that it impedes the silicone layer from becoming detached from the measuring surface (e.g., the diaphragm) of the optical sensor due to the shear forces exerted on the composition. In some implementations, the silicone composition adhesion strength is such that the composition can withstand a maximum load between about 120N to about 500 N; in some adaptations the strength permits withstanding load of about 160N to about 340N. In further implementations, the strength permits the composition to withstand a maximum load between about 210N and about 290N. In other implementations, the silicone composition is configured to have an adhesion strength such that the composition can withstand a maximum load of about 250N.

Generally, the silicone composition is configured to have an adhesion strength such that the composition can withstand a maximum load that is greater than some threshold value. In some implementations, the composition adhesion strength threshold value is about between about 50N and about 150N. In other implementations, the composition adhesion strength threshold value is between about 75N and about 125N. In certain implementations, the composition adhesion strength threshold value is about 100N.

The viscosity should also be configured such that the composition is easily handled during manufacturing and capable of providing protection to the sensor while not being susceptible to overflowing during operation of the blood pump assembly. In some implementations, the viscosity of the composition is between about 2,000 cP and about 8,000 cP. In further implementations, the viscosity of the composition is between about 3,000 cP and about 7,000 cP. In certain implementations, the viscosity of the composition is between about 4,000 cP and about 6,000 cP. In additional implementations, the viscosity of the composition is about 5,000 cP. In other implementations, the viscosity of the composition is between about 2,400 cP and about 7,000 cP.

The viscosity of each silicone component can be considered in addition to the viscosity of each silicone mixture and the viscosity of the final composition overall. For example, in some implementations, the first silicone component and the second silicone component are configured to have viscosities between about 20,000 cP and about 50,000 cP. In other implementations, the first silicone component and the second silicone component are configured to have viscosities between about 25,000 cP and about 45,000 cP. In certain implementations, the first silicone component and the second silicone component are configured to have viscosities between about 30,000 cP and about 40,000 cP. In further implementations, the first silicone component and the second silicone component are configured to have viscosities of about 35,000 cP. The plasticizer has a lower viscosity than the first silicone mixture and the second silicone mixture. Thus, the addition of the plasticizer to the first silicone component and to the second silicone component results in a silicone mixture with a viscosity that is less than the viscosity of the respective component to which the plasticizer is added. In certain implementations, the plasticizer is configured to have a viscosity between about 100 cP and about 500 cP. In further implementations, the plasticizer is configured to have a viscosity between about 200 cP and about 400 cP. In some implementations, the plasticizer is configured to have a viscosity of about 300 cP. The plasticizer may further be configured to have a viscosity that is less than about 300 cP. In certain implementations, the plasticizer is configured to have a viscosity that is less than 200 cP. The viscosity of the plasticizer correlates with the molecular weight of the plasticizer, such that a plasticizer having a lower molecular weight has a lower viscosity than a plasticizer having a larger molecular weight. For example, the plasticizer may be configured so that its molecular weight is in accordance with the above-specified ranges.

In certain implementations, the first silicone mixture and the second silicone mixture are configured to have viscosities between about 2,000 cP and about 5,000 cP. In further implementations, the first silicone mixture and the second silicone mixture are configured to have viscosities between about 3,000 cP and about 4,000 cP. In some implementations, the first silicone mixture and the second silicone mixture are configured to have viscosities of about 3,500 cP.

In some implementations, the plasticizer is configured such that its molecular weight provides a viscosity of the silicone composition that is less than a threshold value. In some implementations, the composition viscosity threshold value is between about 3,000 cP and about 4,000 cP. In certain implementations, the composition viscosity threshold value is between about 3,250 cP and about 3,750 cP. In other implementations, the composition viscosity threshold value is about 3,500 cP. The separate addition of the plasticizer to the first silicone component and to the second silicone component prevents the first silicone component from reacting with the second silicone component to form an inhomogeneous mixture, which is undesirable. As such, the separate addition of the plasticizer to the first and third components helps to form the composition so it has a viscosity below the appropriate threshold values given above.

The rigidity should also be configured to allow the silicone to provide sufficient protection to the measuring surface of the sensor while the blood pump is deployed within the vasculature of the patient while additionally allowing the silicone to be handled during manufacturing. The composition is configured to have a rigidity that is greater than a threshold value. At least one advantage of the rigidity of the composition being above the threshold value is that the rigidity allows the composition to be compatible with existing manufacturing processes. Further, the rigidity being above the threshold value makes the composition easier to apply to the optical sensor. In some implementations, the composition rigidity threshold value is between about 0.5N and about 1.5N. In other implementations, the composition rigidity threshold value is between about 0.75N and about 1.25N. In certain implementations, the composition rigidity threshold value is about 1N.

Additionally, the silicone composition may be configured such that the tackiness allows the composition to adhere to the measuring surface of the optical sensor. Specifically, the composition is configured to have a tack energy below a certain threshold value, below which the tack energy of the composition both provides sufficient adhesion to the sensor and also allows for ease of handling in manufacturing. The tackiness of a substance can be measured by prodding the substance with a probe and determining the energy required to break the bond formed between the substance and the probe. Tackier substances have larger tack energies. In some implementations, the tack energy per unit area of the composition has a minimum value between about 3,500 J/cm² and about 7,500 J/cm². In other implementations, the tack energy per unit area of the composition has a minimum value between about 4,500 J/cm² and about 6,500 J/cm². In further implementations, the tack energy per unit area of the composition has a minimum value of about 5,400 J/cm².

The manufacturing process for the composition may also involve curing of the silicone composition. Specifically, the curing process helps to increase the rigidity, adhesion strength, and viscosity of the composition. In general, the silicone composition may be cured after its application to the sensor. The silicone composition may be cured over a period of time such that a certain percentage of the composition is completely cured, allowing the remainder of the composition to cure by a residual reaction. In some implementations, the period of time over which the composition is cured causes between about 85 percent and about 100 percent of the composition to be completely cured. In such implementations, between about 15 percent and about 0 percent of the composition cures by a residual reaction. In other implementations, the period of time over which the composition is cured causes between about 90 percent and about 95 percent of the composition to be completely cured. In such implementations, between about 10 percent and about 5 percent of the composition cures by a residual reaction. In certain implementations, the period of time over which the composition is cured causes between about 92 percent and about 94 percent of the composition to be completely cured. In such implementations, between about 8 percent and about 6 percent of the composition cures by a residual reaction. In certain implementations, the period of time over which the composition is cured is between about 1 hour and about 9 hours. In other implementations, the period of time over which the composition is cured is between about 3 hours and about 7 hours. In certain implementations, the period of time over which the composition is cured is about 5 hours.

In some implementations, the composition is cured at a temperature of between about 100 degrees Celsius and about 200 degrees Celsius. In certain implementations, the composition is cured at a temperature between about 125 degrees Celsius and about 175 degrees Celsius. In further implementations, the composition is cured at about 150 degrees Celsius. The curing temperature and curing time period are selected in combination such that the desired percentage of the composition is completely cured after being cured over the time period at the curing temperature. At least one advantage of leaving a portion of the composition to cure by a residual reaction is that the manufacturing process is expedited compared to a process in which the entire composition must be actively cured, as it is not necessary to wait for the entire composition to cure completely.

The curing process also configures the silicone composition with the desired shelf life and the desired pot life. In some implementations, the silicone composition is configured to have a shelf life between about 12 months and about 14 months. In other implementations, the silicone composition is configured to have a shelf life of about 13 months. In some implementations, the silicone composition is further configured to have a pot life between about 4 hours and about 10 hours. In other implementations, the silicone composition is configured to have a pot life between about 5 hours and about 9 hours. In some implementations, the silicone composition is configured to have a pot life between about 6 hours and about 8 hours. In certain implementations, the silicone composition is configured to have a pot life of about 7 hours.

The length of time over which the first silicone component and the plasticizer are mixed, as well as the rate at which they are mixed, may be adjusted to yield the desired mechanical properties of the first silicone mixture. In some implementations, the first silicone component and plasticizer are mixed for between about 10 seconds and about 3 minutes in order to create the first silicone mixture. In other implementations, the first silicone component and the plasticizer are mixed for between about 70 seconds and about 110 seconds. In other implementations, the first silicone component and the plasticizer are mixed for between about 80 seconds and about 100 seconds. In further implementations, the first silicone component and the plasticizer are mixed for about 90 seconds. In certain implementations, the first silicone component and the plasticizer are mixed at a rate between 600 rpm and about 2,000 rpm. In other implementations, the first silicone component and the plasticizer are mixed at a rate between 1,000 rpm and about 1,600 rpm. In other implementations, the first silicone component and the plasticizer are mixed at a rate of about 1,300 rpm.

In certain implementations, the second silicone component and the plasticizer are mixed for between about 10 seconds and about 3 minutes to create the second silicone mixture. In some implementations, the second silicone component and the plasticizer are mixed for between about 70 seconds and about 110 seconds. In further implementations, the second silicone component and the plasticizer are mixed for between about 80 seconds and about 100 seconds. In certain implementations, the second silicone component and the plasticizer are mixed for about 90 seconds. In some implementations, the second silicone component and the plasticizer are mixed at a rate between about 600 rpm and about 2,000 rpm. In other implementations, the second silicone component and the plasticizer are mixed at a rate between 1,000 rpm and about 1,600 rpm. In certain implementations, the second silicone component and the plasticizer are mixed at a rate of about 1,300 rpm.

The first silicone mixture and the second silicone mixture are mixed to create the final composition. In some implementations, the first silicone mixture and the second silicone mixture are mixed for between about 10 seconds and about 3 minutes. In some implementations, the first silicone mixture and the second silicone mixture are mixed for between about 70 seconds and about 110 seconds. In further implementations, the first silicone mixture and the second silicone mixture are mixed for between about 80 seconds and about 100 seconds. In other implementations, the first silicone mixture and the second silicone mixture are mixed for about 90 seconds. In certain implementations, the first silicone mixture is mixed with the second silicone mixture at a rate between about 600 rpm and about 2,000 rpm. In other implementations, the first silicone mixture is mixed with the second silicone mixture at rate between about 1,000 rpm and 1,600 rpm. In further implementations, the first silicone mixture is mixed with the second silicone mixture at about 1,300 rpm.

After the first silicone mixture is mixed with the second silicone mixture, the composition is vacuum degassed. In some implementations, the composition is degassed at about room temperature. In other implementations, the composition is degassed at about 22 degrees Celsius. In further implementations, the composition is degassed at about 25 degrees Celsius. The composition may be vacuum degassed for between about 30 minutes and about 50 minutes. In other implementations, the silicone composition is vacuum degassed for about 40 minutes.

According to another implementation, a blood pump assembly as described above comprises an optical sensor assembly. The optical sensor assembly is bound to the pump housing, and the optical sensor assembly contains an optical sensor having a measuring surface. In some implementations, the optical sensor assembly comprises a visor surrounding a support jacket (e.g., a polymer tube or another elongate body having an inner surface, an outer surface, and defining a cavity), the support jacket defining a cavity into which the optical sensor is inserted. In certain implementations, the support jacket comprises polyimide. The specific material of which the support jacket is made can be selected to yield certain mechanical properties of the support jacket and to allow for ease of handling of the polymer during manufacturing. In some implementations, the visor comprises a metal. The metal may comprise stainless steel. Similarly, the metal of which the visor is made can be selected to both yield specific mechanical properties and to ensure sufficient adhesion of the visor to the pump housing of the blood pump assembly. A silicone composition coats the measuring surface of the optical sensor in order to protect the optical sensor from damage caused by the shear forces exerted on the optical sensor by the blood of a patient during the introduction and operation of the blood pump assembly within the patient. The silicone composition that is placed over the optical sensor is configured to be cured. The silicone composition may be configured to be cured within the cavity. The curing of the silicone composition that has been placed on the optical sensor within the cavity helps to expedite the manufacturing process, as only one curing step need be implemented to cure the entirety of the composition in the cavity. In some implementations, the visor is bound to the pump housing by a glue. In certain implementations, the glue may be an epoxy. For example, the glue may be a two-part epoxy. In other implementations, the glue may be a UV light-bonded epoxy. In other implementations, the visor may be fused to the housing. In some implementations, the optical sensor is a silicone optical sensor. In some embodiments, the silicone composition may be, for example, a silicone gel.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1A shows an illustrative blood pump assembly having an optical sensor assembly;

FIG. 1B shows an illustrative interface between a blood pump housing and an optical sensor assembly;

FIG. 2 shows an illustrative optical sensor assembly for use in a blood pump assembly;

FIG. 3 shows an illustrative method of manufacturing a packaging for an optical sensor for use in a blood pump assembly; and

FIG. 4 shows an illustrative method of manufacturing a silicone composition for an optical sensor for use in a blood pump.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, method, and devices disclosed herein, certain illustrative implementations will be described. Although the implementations and features described herein are specifically described for use in connection with a blood pump assembly, it will be understood that the teaching may be adapted and applied to other pumps and other types of medical devices.

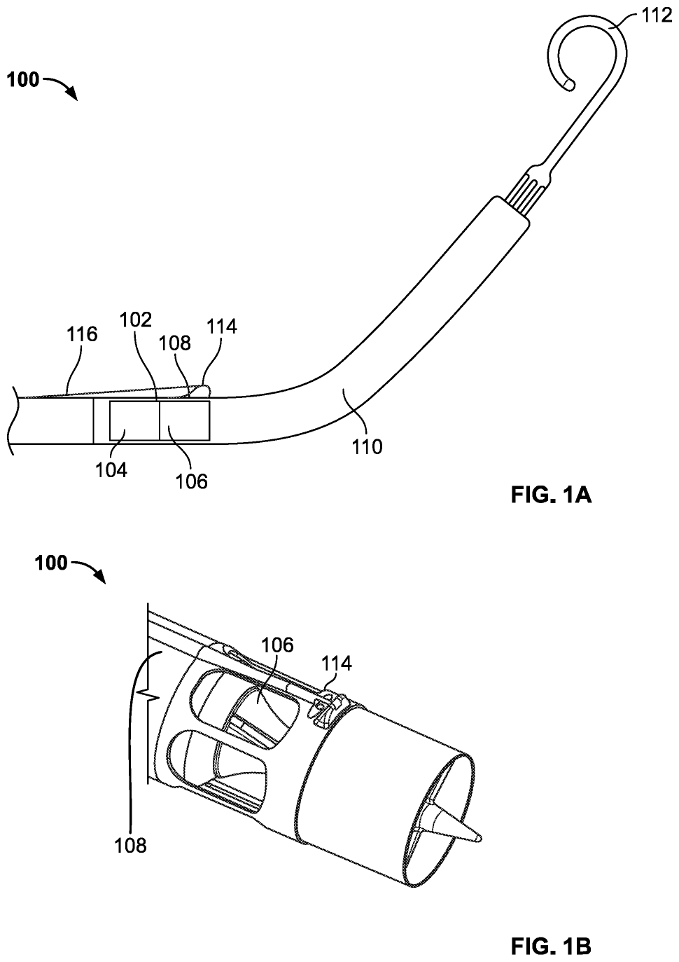

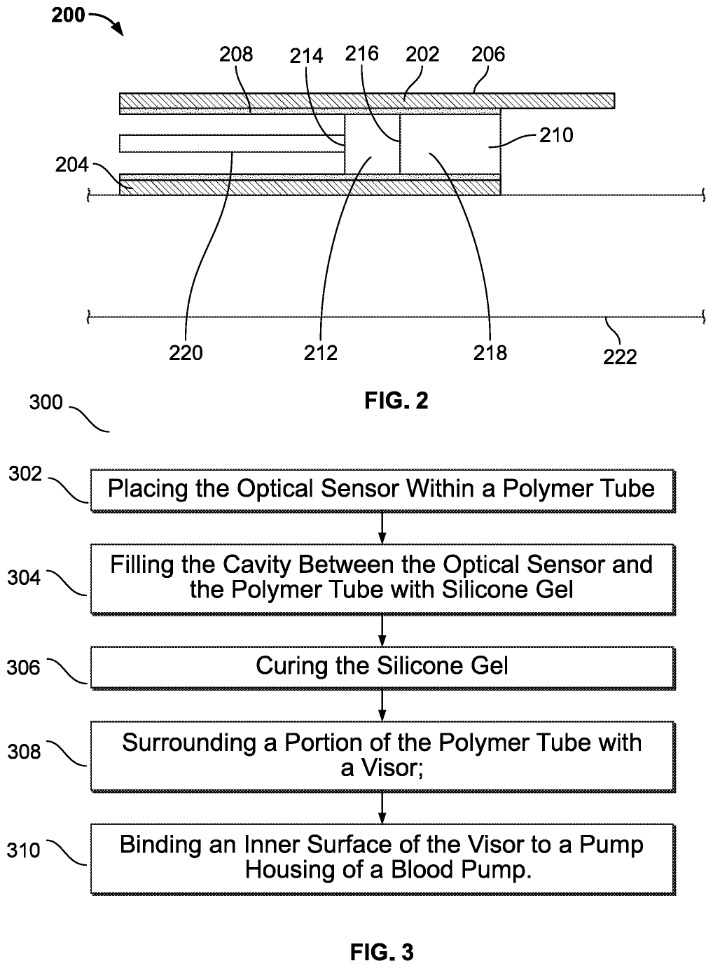

FIG. 1A shows an illustrative blood pump assembly 100 having a pump 102, a motor 104, a rotor 106, pump housing 108, a cannula 110, an atraumatic extension 112, and an optical sensor assembly 114. Optical sensor assembly 114, as described further in relation to FIG. 2 below, comprises a visor, a support jacket, an optical sensor, at least one layer of a silicone composition, and an optical fiber 116. Pump 102 comprises motor 104 and rotor 106. Rotor 106 has at least one blade for conveying fluid through pump 102. Pump housing 108 is configured to surround the at least one blade of rotor 106. Cannula 110 extends from pump housing 108 in a distal direction. Atraumatic extension 112 extends from cannula 110 in a distal direction. In certain implementations, atraumatic extension 112 is a pigtail. Optical sensor assembly 114 is configured to bind to pump housing 108 by the visor of the optical sensor assembly.

FIG. 1B shows an illustrative interface between pump housing 108 and optical sensor assembly 114. The means of adhesion between optical sensor assembly 114 and pump housing 108 is selected in order to tailor the strength of the bond between optical sensor 114 and pump housing 108. The bond strength is advantageously selected based on the shear forces that will be exerted on the pump by the blood during operation and insertion of the pump. For example, a weak bond between optical sensor assembly 114 and pump housing 108 can cause the two components to separate when subject to shear forces that exceed the bond strength. In some implementations, the visor of optical sensor assembly 114 is bound to pump housing 108 by a glue. In certain implementations, the glue is a 2-part epoxy. In other implementations, the glue is a UV light-bonded glue. In further implementations, the visor is fused to pump housing 108. In some implementations, the epoxy used to bond the visor to pump housing 108 is selected based on the tackiness of the epoxy, with larger values of tackiness corresponding to stronger bonds between the visor and pump housing 108. The tackiness of a substance can be measured by prodding the substance with a probe and determining the energy required to break the bond formed be between the substance and the probe. Such measurements yield tack energies of a substance, with larger tack energies corresponding to tackier substances that form bonds that require more energy to break. In certain implementations, the tack energy of the epoxy is between about 2 J/cm² and about 10 J/cm². In other implementations, the tack energy of the epoxy is between about 4 J/cm² and about 8 J/cm². In further implementations, the tack energy of the epoxy is about 6 J/cm². Additionally, a larger amount of a given epoxy used to bind the visor to pump housing 108 corresponds to a stronger bond between the visor and pump housing 108. The optical sensor assembly may be further welded to pump housing 108. Additionally, the visor may be alternately glued to pump housing 108 and welded to pump housing 108 in different regions along the area of the visor. At least one advantage of the configuration of the visor to be glued or fused to pump housing 108 is that both methods of adhesion can be employed in order to provide the strongest bond between the visor and pump housing 108.

FIG. 2 shows an illustrative optical sensor assembly 200 for use in a blood pump assembly (e.g. blood pump assembly 100 of FIG. 1). Optical sensor assembly 200 comprises a visor 202 having a visor inner surface 204 and a visor outer surface 206, a support jacket 208 defining a cavity 210, an optical sensor 212 having an optical sensor first surface 214 and an optical sensor second surface 216, silicone composition 218, an optical fiber 220, and a pump housing 222. Visor outer surface 206 and visor inner surface 204 are configured to surround support jacket 208. In some implementations, the support jacket comprises a polymer tube. Optical sensor 212 is disposed within cavity 210 defined by support jacket 208. Optical sensor has a first surface 214 and a second surface 216. Depending on the orientation of sensor 212 within cavity 210, first surface 214 may be a distal surface or an inner surface. Similarly, second surface 216 may be a proximal surface or an outer surface. In some implementations, first surface 214 is connected to optical fiber 220. In other implementations, first surface 214 is connected to visor inner surface 204. In certain implementations, second surface 216 is configured to receive silicone composition 218. As previously discussed, visor inner surface 204 is configured to bond to pump housing 222 of the blood pump assembly. The pump housing may also be, for example, pump housing 108 of FIG. 1. In some implementations, visor inner surface 204 is bonded to pump housing 222 of the blood pump assembly by a glue. For example, the glue may be an epoxy. The glue may be a 2-part epoxy, or a UV light-curable epoxy. In other implementations, visor inner surface 204 is fused to pump housing 222 of the blood pump assembly. The means of adhesion between visor inner surface 204 and pump housing 222 of the blood pump assembly are selected such that the bond between visor inner surface 204 and pump housing 222 can withstand the shear forces exerted on the blood pump assembly by the blood during the insertion and operation of the blood pump assembly within a patient. As discussed above, the specific means of adhesion between the visor inner surface 204 and pump housing 222 of the blood pump assembly are varied in order to yield the strongest bond between visor inner surface 204 and pump housing 222. For example, in some implementations, the amount of epoxy, and the tackiness of the epoxy used to bond the elements together are selected to ensure a bond with a given adhesion strength. In other implementations, visor inner surface 204 is welded to pump housing 222. As previously discussed, at least one advantage of the configuration of visor inner surface 204 to be glued or fused to pump housing 222 is that both methods of adhesion can be employed in order to provide the strongest bond between the visor inner surface 204 and pump housing 222.

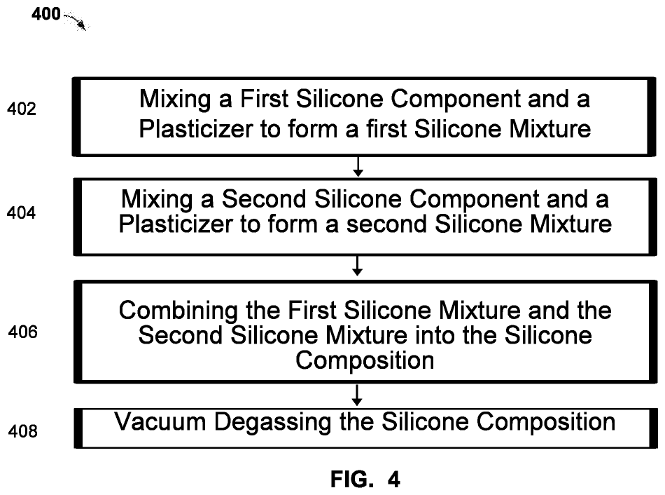

Cavity 210 defined by support jacket 208 prevents the contamination of visor outer surface 206 by silicone composition 218 (e.g., the silicone composition made by the method of FIG. 4). Additionally, the shape of cavity 210 defined by support jacket 208 is configured to accommodate a specific amount of silicone composition 218. Different amounts of silicone composition provide different amounts of protection to the optical sensor of the blood pump assembly. Different amounts of protection to the optical assembly are necessitated by varying magnitudes of shear forces exerted on the optical sensor assembly during the introduction and operation of the pump. The shear forces exerted on the optical sensor assembly during the introduction and operation of the pump may vary in magnitude based on the anatomy of a patient. For example, in obese patients, the blood vessel into which the blood pump assembly must be inserted is further below the surface of the skin that the same blood vessel is in a patient of a healthy weight. This necessitates that, after the blood pump assembly is inserted at the surface of the skin, the blood pump assembly is angled to align with the blood vessel. As such, the insertion of a blood pump assembly into an obese patient places greater strain on the blood pump assembly than does insertion of the same assembly into a patient of a healthy weight, wherein the insertion angle allows the blood pump assembly to be introduced into the blood vessel more directly.

In order to account for the varying forces that may be exerted on a blood pump assembly, the size of the support jacket and the cavity are adjusted in order to sufficiently protect the optical sensor with the appropriate amount of silicone composition. Cardiac procedures that exert larger forces on the optical sensor assembly can be completed with optical sensor assemblies having larger polymer tubes, which accommodate larger amounts of silicone composition 218 to provide the sensor with additional protection. Conversely, cardiac procedures in pediatric patients having smaller anatomies can be completed with optical sensor assemblies having smaller polymer tubes, which allow for the application of additional layers of silicone to the surface of the optical sensor while simultaneously minimizing damage done unto the smaller vasculatures of pediatric patients by larger pumps.

The length or radius of support jacket 208 can be adjusted to change to volume of cavity 210 defined by support jacket 208. Generally, support jackets having larger lengths correspond to larger cavity volumes. As discussed previously, in some implementations, cavity 210 defined by support jacket 208 has a length between about 1 centimeter and about 5 centimeters. In other implementations, cavity 210 has a length between about 2 centimeters and about 4 centimeters. In certain implementations, cavity 210 has a length of about 3 centimeters. Further, a cavity 210 having a larger radius corresponds to a larger cavity volume. In some implementations, cavity 210 has a radius between about 0.1 millimeters and about 0.25 millimeters. In other implementations, the cavity 210 has a radius between about 0.15 millimeters and about 0.20 millimeters. In further implementations, the radius of the cavity 210 is about 0.175 millimeters. For a given length of the cavity 210, a certain volume of cavity 210 may be filled with silicone composition 218. The portion of the volume of cavity 210 that is filled with silicone composition 218 can be selected in order to yield the desire protection to the optical sensor and also to yield a desired manufacturing time, as larger volumes of silicone composition require longer curing times. In certain implementations, between about 30 percent and about 90 percent of the cavity is filled with silicone composition. In other implementations, between about 50 percent and about 70 percent of the cavity is filled with silicone composition. In further implementations, about 60 percent of the cavity is filled with silicone composition.

FIG. 3 shows an illustrative method 300 of manufacturing a packaging for an optical sensor for use in a blood pump assembly. In step 302 of method 300, an optical sensor (e.g. optical sensor 212 of FIG. 2) is placed within a support jacket (e.g., polymer tube polymer tube 208 of FIG. 2), the support jacket configured to define a cavity (e.g. cavity 210 of FIG. 2). In step 304, the cavity is filled between the optical sensor and the support jacket with silicone composition. The silicone composition is subsequently cured in step 306. In step 308, a portion of the support jacket is surrounded with a visor, and in step 310, an inner surface of the visor is bound to a pump housing of the blood pump assembly (e.g. pump housing 108 of blood pump assembly 100 of FIG. 1 or pump housing 222 of FIG. 2). In some implementations, the optical sensor placed within the support jacket is a silicone optical sensor. In certain implementations, the polymer tube comprises polyimide. In further implementations, the visor comprises a metal. The metal may comprise stainless steel or another metal such that the visor has the desired mechanical properties and allows for ease of handling during manufacturing. In certain implementations, the visor is bound to the pump housing by a glue. In some implementations, the glue is an epoxy. In other implementations, the glue is a 2-part epoxy. In further implementations, the glue is a UV light-bonded epoxy.

FIG. 4 shows an illustrative method 400 of manufacturing a silicone composition for use in an optical sensor assembly, the optical sensor assembly for use in a blood pump assembly. Step 402 comprises mixing a first silicone component and a plasticizer to form a first silicone mixture. In some embodiments, the first silicone component and the plasticizer are different materials. In some embodiments, at least one of the first silicone component and the plasticizer is NuSil MED4088. In further embodiments, both the first silicone component and the plasticizer are NuSil MED4088. In some embodiments, the plasticizer is a silicone oil plasticizer. In certain embodiments, the plasticizer is NuSil MED360. The length of time over which the first silicone component and the plasticizer are mixed, as well as the rate at which they are mixed, are adjusted to yield the desired mechanical properties of the first silicone mixture. In some embodiments, the first silicone component and plasticizer may be mixed for between about 10 seconds and about 3 minutes in order to create the first silicone mixture. In other embodiments, the first silicone component and the plasticizer are mixed for between about 70 seconds and about 110 seconds. In other embodiments, the first silicone component and the plasticizer are mixed for between about 80 seconds and about 100 seconds. In further embodiments, the first silicone component and the plasticizer are mixed for about 90 seconds. In certain embodiments, the first silicone component and the plasticizer are mixed at a rate between 600 rpm and about 2,000 rpm. In other embodiments, the first silicone component and the plasticizer are mixed at a rate between 1,000 rpm and about 1,600 rpm. In other embodiments, the first silicone component and the plasticizer are mixed at a rate of at about 1,300 rpm.

Step 404 comprises mixing a second silicone component with the plasticizer to form a second silicone mixture. In certain embodiments, the second silicone component and the plasticizer are mixed for between about 10 seconds and about 3 minutes to create the second silicone mixture. In some embodiments, the second silicone component and the plasticizer are mixed for between about 70 seconds and about 110 seconds. In further embodiments, the second silicone component and the plasticizer are mixed for between about 80 seconds and about 100 seconds. In certain embodiments, the second silicone component and the plasticizer are mixed for about 90 seconds. In some embodiments, the second silicone component and the plasticizer are mixed at a rate between about 600 rpm and about 2,000 rpm. In other embodiments, the second silicone component and the plasticizer are mixed at a rate between 1,000 rpm and about 1,600 rpm. In certain embodiments, the second silicone component and the plasticizer are mixed at a rate of about 1,300 rpm. In some embodiments, the first silicone component, the second silicone component, and the plasticizer are different from each other.

In step 406, the first silicone mixture and the second silicone mixture are subsequently mixed together to yield a silicone composition. In some embodiments, the first silicone mixture and the second silicone mixture are mixed for between about 10 seconds and about 3 minutes. In some embodiments, the first silicone mixture and the second silicone mixture are mixed for between about 70 seconds and about 110 seconds. In further embodiments, the first silicone mixture and the second silicone mixture are mixed for between about 80 seconds and about 100 seconds. In other embodiments, the first silicone mixture and the second silicone mixture are mixed for about 90 seconds. In certain embodiments, the first silicone mixture is mixed with the second silicone mixture at a rate between about 600 rpm and about 2,000 rpm. In other embodiments, the first silicone mixture is mixed with the second silicone mixture at rate between about 1,000 rpm and 1,600 rpm. In further embodiments, the first silicone mixture is mixed with the second silicone mixture at about 1,300 rpm.

The silicone composition is then vacuum degassed in step 408 such that the composition is then configured to protect a measuring surface of an optical sensor for use in the blood pump assembly from the shear forces exerted on the sensor by blood during percutaneous insertion or operation of the blood pump assembly within the patient. In some embodiments, the composition is degassed at about room temperature. In other embodiments, the composition is degassed at about 22 degrees Celsius. In further implementations, the composition is degassed at about 25 degrees Celsius. In some embodiments, the composition is vacuum degassed for between about 30 minutes and about 50 minutes. In other embodiments, the silicone composition is vacuum degassed for about 40 minutes.

The above steps yield a silicone composition with the above-described mechanical properties such that the composition is suitable for use in an optical sensor assembly for a blood pump. As previously stated, the silicone composition may be configured to have an adhesion strength such that the composition can withstand a maximum load between about 160N and about 340N. In further embodiments, the silicone composition is configured to have an adhesion strength such that the composition can withstand a maximum load between about 210N and about 290N. In other embodiments, the silicone composition is configured to have an adhesion strength such that the composition can withstand a maximum load of about 250N. Generally, the silicone composition is configured to have an adhesion strength such that the composition can withstand a maximum load that is greater than some threshold value. In some embodiments, the composition adhesion strength threshold value is about between about 50N and about 150N. In other embodiments, the composition adhesion strength threshold value is between about 75N and about 125N. In certain embodiments, the composition adhesion strength threshold value is about 100N.

Further, the first silicone component and the second silicone component may be configured to have viscosities between about 20,000 cP and about 50,000 cP. In other embodiments, the first silicone component and the second silicone component are configured to have viscosities between about 25,000 cP and about 45,000 cP. In certain embodiments, the first silicone component and the second silicone component are configured to have viscosities between about 30,000 cP and about 40,000 cP. In further embodiments, the first silicone component and the second silicone component are configured to have viscosities of about 35,000 cP. The addition of the plasticizer to the first silicone component and to the second silicone component results in a silicone mixture with a viscosity that is less than the viscosity of the respective component to which the plasticizer is added. In certain embodiments, the first silicone mixture and the second silicone mixture are configured to have viscosities between about 2,000 cP and about 5,000 cP. In further embodiments, the first silicone mixture and the second silicone mixture are configured to have viscosities between about 3,000 cP and about 4,000 cP. In some embodiments, the first silicone mixture and the second silicone mixture are configured to have viscosities of about 3,500 cP.

Additionally, as previously discussed, the plasticizer may be configured such that its molecular weight provides the plasticizer with a viscosity between about 100 cP and about 250 cP. In further embodiments, the plasticizer may be configured such that its molecular weight provides the plasticizer with a viscosity between about 125 cP and about 225 cP. In other embodiments, the plasticizer may be configured such that its molecular weight provides the plasticizer with a viscosity between about 150 cP and about 200 cP. In certain embodiments, the plasticizer may be configured such that its molecular weight provides the plasticizer with a viscosity of about 175 cP. The plasticizer may be configured such that its molecular weight provides a viscosity of the silicone composition that is less than a threshold value. In some implementations, the composition viscosity threshold value is between about 3,000 cP and about 4,000 cP. In certain embodiments, the composition viscosity threshold value is between about 3,250 cP and about 3,750 cP. In other embodiments, the composition viscosity threshold value is about 3,500 cP.

Further, the composition is configured to have a rigidity that is greater than a threshold value, the threshold value being a value above which the rigidity of the composition allows for ease of handling in manufacturing. In some embodiments, the composition rigidity threshold value is between about 0.5N and about 1.5N. In other embodiments, the composition rigidity threshold value is between about 0.75N and about 1.25N. In certain embodiments, the composition rigidity threshold value is about 1N. The composition is also configured to have a tack energy below a certain threshold value, the threshold value being a value below which the tack energy of the composition both provides sufficiently adheres to the sensor and also allows for ease of handling in manufacturing. In some embodiments, the tack energy per unit area of the composition has a minimum value between about 3,500 J/cm² and about 7,500 J/cm². In other embodiments, the tack energy per unit area of the composition has a minimum value between about 4,500 J/cm² and about 6,500 J/cm². In further embodiments, the tack energy per unit area of the composition has a minimum value of about 5,400 J/cm².

In some embodiments, the silicone composition is configured to be cured after application of the composition to the sensor. The silicone composition is cured over a period of time such that a certain percentage of the composition is completely cured, allowing the remainder of the composition to cure by a residual reaction. In some embodiments, the period of time over which the composition is cured causes between about 85 percent and about 100 percent of the composition to be completely cured. In such embodiments, between about 15 percent and about 0 percent of the composition cures by a residual reaction. In other embodiments, the period of time over which the composition is cured causes between about 90 percent and about 95 percent of the composition to be completely cured. In such embodiments, between about 10 percent and about 5 percent of the composition cures by a residual reaction. In certain embodiments, the period of time over which the composition is cured caused between about 92 percent and about 94 percent of the composition to be completely cured. In such embodiments, between about 8 percent and about 6 percent of the composition cures by a residual reaction. In certain embodiments, the period of time over which the composition is cured is between about 1 hour and about 9 hours. In other embodiments, the period of time over which the composition is cured is between about 3 hours and about 7 hours. In certain embodiments, the period of time over which the composition is cured is about 5 hours.

As previously discussed, the curing process also configures the silicone composition with the desired shelf life and the desired pot life. In some embodiments, the silicone composition is configured to have a shelf life between about 12 months and about 14 months. In other embodiments, the silicone composition is configured to have a shelf life of about 13 months. In some embodiments, the silicone composition is further configured to have a pot life between about 4 hours and about 10 hours. In other embodiments, the silicone composition is configured to have a pot life between about 5 hours and about 9 hours. In some embodiments, the silicone composition is configured to have a pot life between about 6 hours and about 8 hours. In certain embodiments, the silicone composition is configured to have a pot life of about 7 hours.

As previously discussed, in some embodiments, the composition is cured at a temperature of between about 100 degrees Celsius and about 200 degrees Celsius. In certain embodiments, the composition is cured at a temperature between about 125 degrees Celsius and about 175 degrees Celsius. In further embodiments, the composition is cured at about 150 degrees Celsius. The curing temperature and curing time period are selected in combination such that the desired percentage of the composition is completely cured after being cured over the time period at the curing temperature. At least one advantage of leaving a portion of the composition to cure by a residual reaction is that the manufacturing process is expedited, as it is not necessary to wait for the entire composition to cure completely.

The foregoing is merely illustrative of the principles of the disclosure and the apparatuses can be practiced by other than the described aspects, which are presented for purposes of illustration and not of limitation. It is to be understood that the apparatuses disclosed herein, while shown for use in percutaneous insertion of blood pumps, may be applied to apparatuses in other applications requiring optical sensors.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above and below, including any components thereof, may be combined or integrated in other systems. Moreover, certain features may be omitted or not implemented.

### Exemplary Implementations

The following examples are given as specific illustrations of the claimed invention. It should be understood, however, that the invention is not limited to the specific details set forth in the following categories of exemplary implementations.

Category A:
A1. An optical sensor assembly for use in a blood pump assembly, the optical sensor assembly comprising:
   a visor having an inner surface and an outer surface;
   a support jacket defining a cavity, wherein
   the support jacket is in contact with the inner surface of the visor;
   an optical sensor having an outer surface and an inner surface, wherein the optical sensor is disposed within the cavity and the inner surface of the optical sensor is in contact with the support jacket; and
   a silicone gel, wherein the silicone gel coats the outer surface of the optical sensor and fills the cavity.
A2. The optical sensor assembly of A1, wherein the support jacket is a polymer tube,
A3. The optical sensor assembly of any of A1-A2, wherein the support jacket is a polyimide tube.
A4. The optical sensor assembly of any of A1-A3, wherein the visor comprises a metal.
A5. The optical sensor assembly of any of A1-A4, wherein the metal is stainless steel.
A6. The optical sensor assembly of any of A1-A5, wherein the visor inner surface is configured to be attached to a pump housing of the blood pump assembly.
A7. The optical sensor assembly of any of A1-A6, wherein the visor inner surface is attached to the pump housing by one of a two-part epoxy, or a UV light-bonded epoxy.
A8. The optical sensor assembly of any of A1-A7, wherein the silicone gel is configured to be cured within the cavity.
A9. The optical sensor assembly of any of A1-A8, wherein the support jacket has an open end and a closed end, and wherein the open end is configured to be closed after the optical sensor is placed within the cavity.
A10. The optical sensor assembly of any of A1-A9, wherein the support jacket has a length between about 1 centimeter and about 5 centimeters.
A11. The optical sensor assembly of any of A1-A10, wherein the support jacket has a length between about 2 centimeters and about 4 centimeters.
A12. The optical sensor assembly of any of A1-A11, wherein the support jacket has a length of about 3 centimeters.
A13. The optical sensor assembly of any of A1-A12, wherein the silicone gel is configured to protect the outer surface of the optical sensor from cracking due to forces exerted on the sensor when the blood pump assembly is used for percutaneous insertion into a patient.

Category B:
B1. An optical sensor assembly for use in a blood pump assembly, the optical sensor assembly comprising:
   a visor having an inner surface and an outer surface;
   a support jacket, having an inner surface and an outer surface, and defining a cavity, wherein the inner surface of the visor is in contact with the outer surface of the support jacket;
   an optical sensor disposed within the cavity, wherein the cavity is filled with a silicone gel, wherein the support jacket confines the silicone gel to flow within the support jacket, and wherein a size of the cavity is configured and an amount of silicone gel is selected to be placed within the support jacket to protect the optical sensor from damage due to forces exerted on the optical sensor during the percutaneous insertion of the blood pump assembly into a patient.
B2. The optical sensor assembly of B1, wherein the support jacket comprises a polymer tube.
B3. The optical sensor assembly of any of B1-B2, wherein the optical sensor is a silicone optical sensor.
B4. The optical sensor assembly of any of B1-B3, wherein the polymer tube comprises polyimide.
B5. The optical sensor assembly of any of B1-B4, wherein the visor comprises a metal.
B6. The optical sensor assembly of any of B1-B5, wherein the metal is stainless steel.
B7. The optical sensor assembly of any of B1-B6, wherein the silicone gel is configured for curing within the cavity.
B8. The optical sensor assembly of any of B1-B7, wherein the silicone gel fills the cavity without contacting an outer surface of the visor, so as to prevent contamination of the outer surface of the visor.
B9. The optical sensor assembly of any of B1-B8, wherein the support jacket prevents the silicone gel from contaminating the outer surface of the visor.

Category C:
C1. A blood pump assembly for insertion into a patient, the blood pump assembly comprising:
   a pump comprising a motor and a rotor, the rotor a blade;
   a pump housing surrounding the blade;
   a cannula extending distal of the pump housing;
   an atraumatic extension extending distally from the cannula; and
   an optical sensor assembly bound to the pump housing by a visor,
   wherein the visor surrounds a support jacket defining a cavity, wherein an optical sensor is disposed within the cavity, and wherein a silicone gel coats the optical sensor within the cavity.
C2. The optical sensor assembly of C1, wherein the optical sensor is a silicone optical sensor.
C3. The optical sensor assembly of any of C1-C2, wherein the support jacket comprises a polymer tube.
C4. The optical sensor assembly of any of C1-C3, wherein the polymer tube is a polyimide tube.
C5. The optical sensor assembly of any of C1-C4, wherein the visor comprises a metal.
C6. The optical sensor assembly of any of C1-05, wherein the metal is stainless steel.
C7. The optical sensor assembly of any of C1-C6, wherein the silicone gel is configured to be cured.
C8. The optical sensor assembly of any of C1-C7, wherein the visor is bound to the pump housing by a glue.
C9. The optical sensor assembly of any of C1-C8, wherein the glue is an epoxy.
C10. The optical sensor assembly of any of C1-C9, wherein the visor is fused to the pump housing.
C11. The optical sensor assembly of any of C1-C10, wherein the support jacket is disposed along the pump housing and within the visor.
C12. The optical sensor assembly of any of C1-C11, wherein the support jacket further comprises an outer surface that is in contact with an outer surface of the pump housing.
C13. The optical sensor assembly of any of C1-C12, wherein the outer surface of the support jacket is in contact with an inner surface of the visor.

Category D:
D1. A method of manufacturing an optical sensor assembly for use in a blood pump assembly, the method comprising:
   placing an optical sensor within a support jacket, the support jacket defining a cavity;
   filling a portion of the cavity between the optical sensor and the support jacket with silicone gel;
   curing the silicone gel;
   surrounding a portion of the support jacket with a visor; and
   binding an inner surface of the visor to a pump housing of a blood pump.
D2. The method of D1, wherein the optical sensor is a silicone optical sensor.
D3. The method of any of D1-D2, wherein the support jacket comprises a polymer tube.
D4. The method of any of D1-D3, wherein the polymer tube comprises polyimide.
D5. The method of any of D1-D4, wherein the visor comprises a metal.
D6. The method of any of D1-D5, wherein the metal is stainless steel.
D7. The method of any of D1-D6, wherein the visor is bound to the pump housing by an epoxy.
D8. The method of any of D1-D7, wherein the epoxy is one of a two-part epoxy or a UV-light curable epoxy.
D9. The method of any of D1-D8, wherein the visor is fused to the pump housing.

Category E:
E1. A method of manufacturing a silicone composition for use in a blood pump assembly, the method comprising:
   mixing a first silicone component and a plasticizer to form a first silicone mixture;
   mixing a second silicone component and the plasticizer to form a second silicone mixture; combining the first silicone mixture and the second silicone mixture into the silicone composition; and
   vacuum degassing the silicone composition, and wherein the composition is configured to protect a measuring surface of an optical sensor for use in the blood pump assembly from shear forces exerted on the sensor by blood during percutaneous insertion of the blood pump assembly into a patient.
E2. The method of E1, wherein the first silicone component is an activator.
E3. The method of any of E1-E2, wherein the second silicone component comprises a platinum-based catalyst.
E4. The method of any of E1-E3, wherein the plasticizer is a silicone oil plasticizer.
E5. The method of any of E1-E4, wherein the ratios of the first and second components to the plasticizer are 1:1 such that the composition has a ratio of the first to the second component to the plasticizer of 1:1:2.
E6. The method of any of E1-E5, wherein an adhesion strength of the silicone composition is configured such that the composition can withstand a maximum load between about 160 N and about 340 N.
E7. The method of any of E1-E6, wherein the adhesion strength of the silicone composition is configured such that the composition can withstand a maximum load between about 210 N and about 290 N.
E8. The method of any of E1-E7, wherein the adhesion strength of the silicone composition is configured such that the composition can withstand a maximum load of about 250 N.
E9. The method of any of E1-E8, wherein the adhesion strength of the silicone composition is configured such that the composition can withstand a maximum load that is greater than about 50 N.
E10. The method of any of E1-E9, wherein the first and second silicone components are configured to have a viscosity between about 30,000 cP and about 40,000 cP.
E11. The method of any of E1-E10, wherein the first and second silicone components are configured to have a viscosity of about 35,000 cP.
E12. The method of any of E1-E11, wherein the first and second silicone mixtures are configured to have a viscosity between about 3,000 cP and about 4,000 cP.
E13. The method of any of E1-E12, wherein the first and second silicone mixtures are configured to have a viscosity of about 3,500 cP.
E14. The method of any of E1-E13, wherein the silicone oil plasticizer is configured to have a low molecular weight such that the silicone composition viscosity is less than 200 cP.
E15. The method of any of E1-E14, wherein adding the plasticizer to the first and third components separately configures the composition to have a viscosity that is less than 300 cP.
E16. The method of any of E1-E15, wherein the silicone composition is configured to have a viscosity between about 2,400 cP and about 7,000 cP.
E17. The method of any of E1-E16, wherein the silicone composition is configured to have a viscosity between about 3,000 cP and about 6,000 cP.
E18. The method of any of E1-E17, wherein the silicone composition is configured to have a viscosity between about 4,000 cP and about 6,000 cP.
E19. The method of any of E1-E18, wherein the silicone composition is configured to have a viscosity of about 5,000 cP.
E20. The method of any of E1-E19, wherein the composition is configured to have a rigidity that is greater than about 1.5 N.
E21. The method of any of E1-E20, wherein the composition is configured to have a rigidity that is greater than about 1.2 N.
E22. The method of any of E1-E21, wherein the composition is configured to have a rigidity that is greater than about 0.9 N.
E23. The method of any of E1-E22, wherein the composition is configured to be cured after application to the sensor.
E24. The method of any of E1-E23, wherein the composition is cured over a period of time such that about 90 to about 95 percent of the composition is cured.
E25. The method of any of E1-E24, wherein the period of time is between about 1 and about 9 hours.
E26. The method of any of E1-E25, wherein the period of time is between about 3 and about 7 hours.
E27. The method of any of E1-E26, wherein the period of time is about 5 hours.
E28. The method of any of E1-E27, wherein the composition is cured at a temperature between about 100 degrees Celsius and about 200 degrees Celsius.
E29. The method of any of E1-E28, wherein the composition is cured at a temperature between about 125 degrees Celsius and about 175 degrees Celsius.
E30. The method of any of E1-E29, wherein the composition is cured at a temperature of about 150 degrees Celsius.
E31. The method of any of E1-E30, wherein the composition is configured such that the amount of silicone used allows the sensor to be protected while also limiting a tackiness of the composition below a threshold tackiness value.
E32. The method of any of E1-E31, wherein the composition has a tackiness such that the minimum load exerted on a probe by the composition is between about −2.1 N and about 0 N.
E33. The method of any of E1-E32, wherein the composition has a tackiness such that the minimum load exerted on a probe by the composition is between about −1.0 N and about 0 N.
E34. The method of any of E1-E33, wherein the composition has a tackiness such that the maximum load exerted on a probe by the composition is about −0.1 N.
E35. The method of any of E1-E34, wherein the threshold tackiness value is configured to be low such that the sensor can adhere to a visor for use in a blood pump assembly.
E36. The method of any of E1-E35, wherein the first and second silicone components and the plasticizer are biocompatible.
E37. The method of any of E1-E36, wherein the ratio of silicone to silicone oil plasticizer is configured to allow for adhesion of the composition to the visor while also having a viscosity that allows for ease of handling.
E38. The method of any of E1-E37, wherein the first silicone component and the plasticizer are mixed for between about 10 seconds and about 3 minutes.
E39. The method of any of E1-E38, wherein the first silicone component and the plasticizer are mixed for about 90 seconds.
E40. The method of any of E1-E39, wherein the first silicone component and the plasticizer are mixed at between about 600 rpm and about 2000 rpm.
E41. The method of any of E1-E40, wherein the first silicone component and the plasticizer are mixed at between about 1000 rpm and about 1600 rpm.
E42. The method of any of E1-E41, wherein the first silicone component and the plasticizer are mixed at about 1300 rpm.
E43. The method of any of E1-E42, wherein the second silicone component and the plasticizer are mixed for between about 10 seconds and about 3 minutes.
E44. The method of any of E1-E43, wherein the second silicone component and the plasticizer are mixed for about 90 seconds.
E45. The method of any of E1-E44, wherein the second silicone component and the plasticizer are mixed at between about 600 rpm and about 2000 rpm.
E46. The method of any of E1-E45, wherein the second silicone component and the plasticizer are mixed at between about 1000 rpm and about 1600 rpm.
E47. The method of any of E1-E46, wherein the second silicone component and the plasticizer are mixed at about 1300 rpm.
E48. The method of any of E1-E47, wherein the first silicone mixture and the second silicone mixture are mixed for between about 10 seconds and about 3 minutes.
E49. The method of any of E1-E48, wherein the first silicone mixture and the second silicone mixture are mixed for about 90 seconds.
E50. The method of any of E1-E49, wherein the first silicone mixture and the second silicone mixture are mixed at between about 600 rpm and about 2000 rpm.
E51. The method of any of E1-E50, wherein the first silicone mixture and the second silicone mixture are mixed at between about 1000 rpm and about 1600 rpm.
E52. The method of any of E1-E51, wherein the first silicone mixture and the second silicone mixture are mixed at about 1300 rpm.
E53. The method of any of E1-E52, wherein the silicone composition is vacuum degassed at room temperature.
E54. The method of any of E1-E53, wherein the silicone composition is vacuum degassed for between about 30 minutes and about 50 minutes.
E55. The method of any of E1-E54, wherein the silicone composition is vacuum degassed for about 40 minutes.
E56. The method of any of E1-E55, wherein the measuring surface is a diaphragm.
E57. The method of any of E1-E56, wherein the silicone composition is configured to have a shelf life between about 12 months and about 14 months.
E58. The method of any of E1-E57, wherein the silicone composition is configured to have a pot life between about 5 hours and about 9 hours.

Category F:
F1. A blood pump assembly comprising:
   a pump, the pump comprising a motor and a rotor, the rotor having at least one blade; a pump housing, the pump housing surrounding the at least one blade of the rotor;
   a cannula;
   an atraumatic extension extending distally from the cannula; and,
   a silicone optical sensor assembly bonded to the pump housing, the sensor assembly comprising an optical sensor having a measuring surface, the measuring surface having a coat of silicone, the coat of silicone comprising a mixture of a first silicone component, a plasticizer, and a second silicone component.
F2. The blood pump assembly of F1, wherein the coat of silicone comprises the composition of any of A1-A54.
F3. The blood pump assembly of any of F1-F2, wherein the optical sensor assembly further comprises a visor and a support jacket.
F4. The blood pump assembly of any of F1-F3, wherein the support jacket defines a cavity in which the optical sensor and the coat of silicone are disposed.
F5. The blood pump assembly of any of F1-F4, wherein the visor radially surrounds the support jacket.

Category G:
G1. A blood pump assembly comprising:
   a pump, the pump comprising a motor and a rotor, the rotor having at least one blade; a pump housing, the pump housing surrounding the at least one blade of the rotor;
   a cannula;
   an atraumatic extension extending distally from the housing; and, a silicone optical sensor comprising a measuring surface,
   wherein the measuring surface is configured to receive a coat of silicone, the coat of silicone comprising a mixture of a first silicone component, a plasticizer, and a second silicone component, and wherein the silicone is configured with at least one of a desired viscosity, rigidity, lap shear, and tackiness.
G2. The blood pump assembly of G1, wherein the coat of silicone comprises the composition of any of E1-E54.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto. All references cited herein are incorporated by reference in their entirety and made part of this application.

1. An optical sensor assembly for use in a blood pump assembly, the optical sensor assembly comprising:
a visor having an inner surface and an outer surface;
a support jacket defining a cavity, wherein the support jacket is in contact with the inner surface of the visor;
an optical sensor having a first surface and a second surface, wherein the optical sensor is positioned within the cavity; and
a silicone composition positioned within the cavity, wherein the silicone composition coats the second surface of the optical sensor.

2. The optical sensor assembly of claim 1, wherein the visor inner surface is configured to be attached to a pump housing of a blood pump assembly.

3. The optical sensor assembly of claim 2, wherein the visor inner surface is attached to the pump housing by an epoxy.

4. The optical sensor assembly of claim 1, wherein the visor comprises a metal.

5. The optical sensor assembly of claim 4, wherein the metal is stainless steel.

6. The optical sensor assembly of claim 1, wherein the support jacket has an open end and a closed end, and wherein the open end of the support jacket is configured to be closed after the optical sensor is positioned within the cavity.

7. The optical sensor assembly of claim 1, wherein the support jacket is a polymer tube.

8. The optical sensor assembly of claim 7, wherein the support jacket is a polyimide tube.

9. The optical sensor assembly of claim 1, wherein the silicone composition is a silicone gel.

10. The optical sensor assembly of claim 1, wherein the silicone composition is configured to be cured within the cavity.

11. The optical sensor assembly of claim 1, wherein the silicone composition is configured to protect the second surface of the optical sensor from damage due to forces exerted on the optical sensor when the blood pump assembly is used for percutaneous insertion into a patient.

12. The optical sensor assembly of claim 1, wherein the cavity is configured to accommodate an amount of the silicone composition selected to protect the optical sensor from damage due to forces exerted on the optical sensor during the percutaneous insertion of the blood pump assembly into a patient.

13. The optical sensor assembly of claim 1, wherein the optical sensor is a silicone optical sensor.

14. A blood pump assembly for insertion into a patient, the blood pump assembly comprising:
a pump comprising a motor and a rotor, the rotor having at least one blade;
a pump housing surrounding the at least one blade;
a cannula extending distal of the pump housing;
an atraumatic extension extending distally from the cannula; and
an optical sensor assembly, wherein the optical sensor assembly comprises:
a visor;
a support jacket defining a cavity;
an optical sensor positioned within the cavity; and
a silicone composition positioned within the cavity, wherein the silicone composition coats the optical sensor, and
wherein the optical sensor assembly is attached to the pump housing by the visor.

15. The blood pump assembly of claim 14, wherein the optical sensor is a silicone optical sensor.

16. The blood pump assembly of claim 14, wherein the silicone composition comprises a mixture of a first silicone component, a plasticizer, and a second silicone component, and wherein the silicone composition has at least one of a desired viscosity, rigidity, lap shear, and tackiness.

17. The blood pump assembly of claim 14, wherein the silicone composition is configured to be cured within the cavity.

18. The blood pump assembly of claim 14, wherein the silicone composition is a silicone gel.

19. The blood pump assembly of claim 14, wherein the silicone composition coats a measuring surface of the optical sensor.

20. The blood pump assembly of claim 14, wherein the visor is attached to the pump housing by a glue.

21. A method of manufacturing an optical sensor assembly for use in a blood pump assembly, the method comprising:
positioning an optical sensor within a support jacket, wherein the support jacket defines a cavity;
positioning a silicone composition within the cavity such that the silicone composition coats a surface of the optical sensor;
curing the silicone composition;
contacting a portion of the support jacket with a visor; and
attaching the visor to a pump housing of a blood pump.

22. The method of claim 21, wherein the silicone composition is configured to protect a measuring surface of the optical sensor for use in a blood pump assembly from shear forces exerted on the optical sensor by blood during percutaneous insertion of the blood pump assembly into a patient.

23. The method of claim 22, wherein the measuring surface is a diaphragm.

24. The method of claim 21, further comprising making the silicone composition, wherein the method of making the silicone composition comprises:
mixing a first silicone component and a plasticizer to form a first silicone mixture;
mixing a second silicone component and the plasticizer to form a second silicone mixture;
combining the first silicone mixture and the second silicone mixture to make the silicone composition; and
vacuum degassing the silicone composition.

25. The method of claim 24, wherein the first silicone component comprises an activator.

26. The method of claim 24, wherein the second silicone component comprises a platinum-based catalyst.

27. The method of claim 24, wherein the plasticizer is a silicone oil plasticizer.

28. The method of claim 24, wherein the first silicone component, the second silicone component, and the plasticizer are biocompatible.

29. The method of claim 24, wherein the first silicone component is a different material than the plasticizer.
